(19) United States
(12) Patent Application Publication
(10) Pub. No.: US 2025/0221780 A1
(43) Pub. Date: Jul. 10, 2025

(54) MEDICAL ROBOT, SURGICAL AUXILIARY POSITIONING SYSTEM AND CONTROL METHOD

(71) Applicant: SHANGHAI DROIDSURG MEDICAL CO., LTD, Shanghai (CN)

(72) Inventors: Wener LV, Shanghai (CN); Jing WU, Shanghai (CN); Shaobai WANG, Shanghai (CN); Qingdong TAO, Shanghai (CN)

(73) Assignee: SHANGHAI DROIDSURG MEDICAL CO., LTD, Shanghai (CN)

(21) Appl. No.: 18/857,209

(22) PCT Filed: Sep. 23, 2022
(86) PCT No.: PCT/CN2022/120916

§ 371 Date: Oct. 16, 2024

(30) Foreign Application Priority Data
Sep. 23, 2021 (CN) 202111116561.4
Jul. 27, 2022 (CN) 202210893408.0

(51) Int. Cl. A61B 34/30

(52) U.S. Cl. CPC A61B 34/30; A61B 2034/305

(57) ABSTRACT

The provided is a medical robot, including single-arm mechanism, which including pitching adjusting mechanism and deflection adjusting mechanism. One end of telescopic pull rod of pitching adjusting mechanism is hinged to one end of hinge portion, other end connects to pitching motor; other end of hinge portion hinges to one end of main rotating shaft to be driven by pitching motor to slide telescopically relative to main rotating shaft to implement pitching movement of hinge portion. First gear of deflection adjusting mechanism meshes with second gear and sleeves outer wall of main rotating shaft, main rotating motor drives first gear by second gear, to drive main rotating shaft to rotate, such that deflection adjusting mechanism implements deflection movement of pitching frame by means of transmission of main rotating shaft of pitching adjusting mechanism. The further provided are surgical auxillary positioning system and control method.

The present application claims the priority to prior application No. 2021111165614, filed on Sep. 23, 2021 and entitled “Surgical Auxillary Positioning System and Control Method therefor”, and also claims the priority to prior application No. 2022108934080, filed on Jul. 27, 2022 and entitled “Medical Robot”. The content of both foregoing applications are incorporated herein by reference.

### TECHNICAL FIELD

The present disclosure relates to the technical field of medical instruments, in particular to a medical robot, a surgical auxiliary positioning system and a control method.

### BACKGROUND TECHNOLOGY

A wide variety of medical robots have appeared in the medical field. For example, a man-machine collaborated medical robot disclosed in Chinese patent of invention with publication No. CN111085990A can compile an operation plan independently, determine an action program according to an actual situation before changing the action into a movement of an operation mechanism. However, in the prior art, in most cases, a medical robot has a large apparatus size and occupies a large space, which is not conducive for an operator to perform a flexible control and operation.

Puncture is a treatment technology of puncturing a body cavity to extract secretions for testing, injecting gas or a contrast medium into the body cavity for a contrast examination, or injecting a medicine into the body cavity. In the prior art, a puncture surgery is generally performed in following two ways: one is that, a doctor obtains a plurality of image information by means of computed tomography (CT), magnetic resonance imaging (MRI), and more, to determine a puncture point and a puncture angle under help of a computer-aided design, before formulating a corresponding surgical solution and performing the puncture; another is that, projecting X-rays in a plurality of different directions, and finding a target point to be punctured, before carrying out a puncture under guidance of the X-rays. Since both ways stated above are mainly depending on a manual surgery according to a solution prepared before the surgery, experience of a doctor and an observed condition, thus a puncture precision is affected by a plurality of factors in various aspects, that increases a risk of the surgery.

Further, in an application of a mechanical arm in an existing medical equipment, it is usually achieving a position adjustment by continuously rotating multiple wrist joints, which may cause a larger error, having a larger operation limitation, or by directional movement in a single direction, which causes a single use pattern and incapacity to cope with a complex surgical condition.

### CONTENT OF THE INVENTION

The present disclosure provides a medical robot capable of reducing an occupied space.

The medical robot of the present disclosure includes a single-arm mechanism, where the single-arm mechanism includes a pitching adjusting mechanism and a deflection adjusting mechanism; the pitching adjusting mechanism includes a pitching motor, a support frame, a main rotating shaft, a telescopic pull rod and a pitching frame, the pitching frame includes a hinge portion, the main rotating shaft is movably connected to the support frame, the telescopic pull rod is located in the main rotating shaft, one end of the telescopic pull rod is hinged to one end of the hinge portion, the other end of the hinge portion is hinged to one end of the main rotating shaft, the other end of the telescopic pull rod is connected to the pitching motor, and the pitching motor is configured to drive the telescopic pull rod to telescopically slide relative to the main rotating shaft so as to implement pitching movement of the hinge portion; and the deflection adjusting mechanism includes a first gear, a second gear and a main rotating motor, the first gear sleeves an outer side of the main rotating shaft, the second gear meshes with the first gear, and the main rotating motor is connected to the second gear and configured to drive the second gear to rotate to drive the first gear to rotate and further to drive the main rotating shaft to rotate to implement deflection movement of the pitching frame.

The medical robot of the present disclosure has the beneficial effects that in the pitching adjusting mechanism, one end of the telescopic pull rod is hinged to one end of the hinge portion, the other end of the telescopic pull rod is connected to the pitching motor, and the other end of the hinge portion is hinged to one end of the main rotating shaft, so as to be driven by the pitching motor to slide telescopically relative to the main rotating shaft to implement pitching movement of the hinge portion. The first gear meshes with the second gear and sleeves an outer side of the main rotating shaft, and the main rotating motor connected to the second gear may drive the first gear by means of the second gear to drive the main rotating shaft to rotate so as to implement deflection movement of the pitching frame, such that the deflection adjusting mechanism implements the deflection movement of the pitching frame by means of transmission of the main rotating shaft of the pitching adjusting mechanism, so as to improve an integration level, and effectively reduce an occupied space.

Preferably, the first gear has a larger outer diameter than the second gear.

Preferably, the support frame includes a first hole and a second hole, and the main rotating shaft penetrates the first hole and the second hole.

Preferably, a first bearing is arranged in the first hole, a second bearing is arranged in the second hole, and the main rotating shaft penetrates the first bearing and the second bearing.

Preferably, the support frame includes a third hole and a fourth hole, and the main rotating motor penetrates the third hole and the fourth hole.

Preferably, the pitching motor is a telescoping motor.

Preferably, one end of the hinge portion includes a first through hole and a first pin that penetrate two opposite walls of the hinge portion, one end of the telescopic pull rod is connected to the first pin, and the first pin is located in the first through hole.

Preferably, one end of the main rotating shaft includes a first triangular plate, a second triangular plate and a connection plate, the first triangular plate and the second triangular plate are arranged oppositely and are connected by means of the connection plate, a fifth hole is provided in the connection plate, the telescopic pull rod penetrates the fifth hole, a sixth hole is provided in one end of the first triangular plate, a seventh hole is provided in one end of the second triangular plate; and the other end of the hinge portion includes a second pin and is provided with a second through hole, the second pin is located between the first triangular plate and the second triangular plate to communicate the sixth hole, the second through hole and the seventh hole sequentially, and the second pin is located in a channel where the sixth hole, the second through hole and the seventh hole are in communication, so as to hinge the hinge portion to one end of the main rotating shaft.

Preferably, the medical robot further includes a cross slide table, where the cross slide table includes a rail mechanism, the rail mechanism includes a first slider, a second slider, a first rail and a second rail, the first rail and the second rail are located on a horizontal plane, the first rail and the second rail are perpendicular to each other, the first slider is slidably connected to the first rail, the first rail is fixedly connected to the second slider, and the second slider is slidably connected to the second rail and configured to drive the first rail to slide along the second rail.

Preferably, the cross slide table further includes a first screw mechanism, the first screw mechanism includes a first electric motor, a first screw and a first nut, the first electric motor is connected to one end of the first screw, the first screw is in threaded connection to the first nut, the first slider is fixedly connected to the first nut, and the first electric motor is configured to drive the first screw to rotate so as to drive the first slider to move along the first rail.

Preferably, the cross slide table further includes a second screw mechanism, the second screw mechanism includes a second electric motor, a second screw and a second nut, the second electric motor is connected to one end of the second screw, the second screw is in threaded connection to the second nut, the first slider is fixedly connected to the second nut, and the second electric motor is configured to drive the second screw to rotate so as to drive the second slider to move along the second rail.

The present disclosure further provides a surgical auxillary positioning system, which is configured to solve the problems that in the prior art, surgical accuracy is not high, harm to patients is large, operation limitation is severe, and use is inflexible.

The surgical auxillary positioning system includes a first adjusting module, a second adjusting module, an information feedback module, a clamping module, a workstation module, a navigation module, an image capturing module and a display module, where the second adjusting module is the single-arm mechanism; the second adjusting module is arranged at one end of the first adjusting module, the first adjusting module is configured to adjust a working area of the second adjusting module, the clamping module is arranged on the second adjusting module, the second adjusting module is configured to adjust a position and an angle of the clamping module, and the clamping module is configured to fix an surgery execution structure; the information feedback module is electrically connected to the workstation module, the information feedback module is arranged on the second adjusting module, and the information feedback module is configured to determine working area information of the second adjusting module and transmit the working area information to the workstation module; the image capturing module is electrically connected to the display module and the workstation module separately, and the image capturing module obtains image information of a surgical object and transmits the image information to the display module and the workstation module; the navigation module is electrically connected to the display module and the workstation module separately, the navigation module is arranged on the clamping module and the surgical object, and the navigation module collects position information of the surgical object and the clamping module and transmits the position information to the display module and the workstation module; the display module is electrically connected to the workstation module, and the display module receives and displays the image information and the position information; the workstation module is electrically connected to the second adjusting module, the workstation module receives the image information and the position information, the workstation module establishes a working path according to the image information, and the workstation module compares the position information with the working path to obtain adjusting information; and the workstation module receives the working area information, and the workstation module outputs an adjusting instruction according to the adjusting information and the working area information to control the second adjusting module to be turned on or off, and determines whether to adjust the first adjusting module according to the adjusting instruction.

The surgical auxillary positioning system of the present disclosure has the beneficial effects that the first adjusting module is manually adjusted, during use, the first adjusting module adjusts the working area of the second adjusting module, the second adjusting module adjusts the position and the angle of the clamping module, the clamping module fixes an instrument needing in surgery, the second adjusting module and the information feedback module are both electrically connected to the workstation module, and the working area is fed back by means of the information feedback module, such that the manual adjustment of the first adjusting module may be conveniently performed when the second adjusting module may not reach a required position; the image capturing module is electrically connected to the display module and the workstation module separately, the navigation module is electrically connected to the display module and the workstation module separately, and then the obtained image information of the surgical object and the obtained position information of the clamping module are displayed, such that an operator may conveniently observe the surgical object; and moreover, the workstation module establishes a working path according to the image information and the position information and obtains the adjusting information by means of comparison, and then the workstation module determines whether to adjust the first adjusting module according to the working area information and the adjusting information and controls the second adjusting module to be turned on or off. By means of the arrangement, the surgical auxillary positioning system in the present application may cover a large area, cope with different surgical scenes, establish a surgical path according to the image information, and adjust the angle under the control of the workstation module. Further, the accuracy is high, errors generated by the operator may be eliminated, entire accuracy is high, and damage to the surgical object is small. Moreover, by means of cooperation of the first adjusting module and the second adjusting module, adjusting precision of the clamping module may be conveniently improved, and an adjusting time may be conveniently shortened.

Optionally, the second adjusting module is a single-arm mechanism of the medical robot according to the present disclosure.

Preferably, the surgical auxillary positioning system further includes an image processing module, where the image processing module is electrically connected to the image capturing module, the display module and the workstation module separately, the image processing module is configured to fuse different image information obtained by the image capturing module, the image processing module transmits fused image information to the image capturing module, the display module and the workstation module. The surgical auxillary positioning system has the beneficial effects that by means of the arrangement, the image information obtained at different moments may be conveniently processed and superposed, such that the operator may conveniently perform adjustment according to a current situation during actual use.

Preferably, the surgical auxillary positioning system further includes an image analysis module, where the image analysis module is connected to the image capturing module, the image processing module, the display module and the workstation module separately, the image processing module or the image capturing module transmits the image information to the image analysis module, the image analysis module receives the image information, the image analysis module analyzes positions of a tissue structure and a focus area in the image information and delineates boundaries of the tissue structure and the focus area, and the image analysis module transmits delineated image information to the workstation module and the display module. The surgical auxiliary positioning system has the beneficial effects that by means of the arrangement, the tissue structure and the focus area in the surgical object may be conveniently marked and may be conveniently observed by the operator, and further, a surgical solution may be conveniently made.

Preferably, the surgical auxiliary positioning system further includes a comparison module, a storage module and a calling module, where the comparison module is electrically connected to the workstation module, the workstation module transmits the delineated image information and the working path to the comparison module, the comparison module receives the delineated image information and the working path, the comparison module is configured to compare the working path with the position of the tissue structure and the position of the focus area separately to obtain a comparison result, and the comparison module transmits the comparison result to the workstation module; the storage module is electrically connected to the workstation module, the workstation module receives the comparison result, the workstation module transmits the working path to the storage module or deletes the working path according to the comparison result, and the storage module receives and stores the working path; and the calling module is electrically connected to the storage module and the workstation module separately, and the calling module calls the working path stored by the storage module and transmits the working path to the workstation module. The surgical auxiliary positioning system has the beneficial effects that by means of the arrangement, the comparison module compares the working path with the image information, and a feasible surgical solution is transmitted to the storage module by means of the workstation module to be stored and then is called by means of the calling module, such that the solution is conveniently made and selected in advance during surgery, so as to shorten the surgery duration in the surgery.

Preferably, the single-arm mechanism includes an execution module, the execution module is arranged on the clamping module, the execution module is connected to the workstation module, and the workstation module is configured to control the execution module to be turned on or off. The single-arm mechanism has the beneficial effects that by means of the arrangement, the execution module replaces the operator to perform the surgery, on one hand, remote operation may be achieved, and on the other hand, errors caused by the operator may be conveniently eliminated.

Preferably, the navigation module includes a reference target and an optical target; and the reference target is arranged on the surgical object, and the optical target is arranged on the clamping module. The navigation module has the beneficial effects that by means of the arrangement, the position information of the surgical object and the execution module is convenient to know, and relative position information of the surgical object and the execution module is further convenient to know, such that the relative position information is convenient to determine when the surgery is executed, and misoperation is avoided.

Preferably, the surgical auxillary positioning system further includes an isolation layer structure arranged outside the first adjusting module and the second adjusting module. The surgical auxillary positioning system has the beneficial effects that by means of the arrangement, the first adjusting module and the second adjusting module may be prevented from polluting the surgical object.

The present disclosure further provides a control method for a surgical auxillary positioning system. The control method includes: S1, arranging the navigation module on the clamping module and the surgical object, determining the position information of the clamping module and the surgical object, collecting the image information by the image capturing module, arranging the information feedback module on the second adjusting module to determine the working area information, transmitting the position information, the image information and the working area information to the workstation module, and transmitting the position information and the image information to the display module; S2, receiving, by the workstation module, the image information, the working area information and the position information, establishing, by the workstation module, a space coordinate system according to the image information, and receiving and displaying, by the display module, the position information and the image information; S3, establishing, by the workstation module, a working path by means of the space coordinate system; S4, comparing, by the workstation module, the position information with the working path to obtain adjustment information; S5, outputting, by the workstation module, an adjusting instruction according to the adjusting information and the working area information; under the condition that a distance indicated by the adjusting information is larger than an area covered by the working area information, adjusting a working area of the second adjusting module by means of the first adjusting module, and controlling the second adjusting module to adjust a direction and a position of the clamping module by means of the workstation module; and under the condition that a distance indicated by the adjusting information is not larger than an area covered by the working area information, controlling the second adjusting module to adjust a direction and a position of the clamping module by means of the workstation module; and S6, working by means of the clamping module.

The control method for a surgical auxillary positioning system has the beneficial effects that the image information of the surgical object is obtained by means of the image capturing module, the position information of the surgical object and the clamping module is obtained by means of the navigation module, the working area information is determined by means of the information feedback module, the display module performs display, the workstation module establishes a working path according to the information and determines whether to adjust the first adjusting module according to the working path and the position information, and then the position of the clamping module is adjusted under the control of the workstation module, such that when the first adjusting module needs no adjustment, an adjusting amplitude of the second adjusting module during adjustment is conveniently shortened, so as to shorten the adjusting time and further improve the adjusting precision of the second adjusting module, the working area of the second adjusting module is manually adjusted by means of the first adjusting module, and a coverage range is wide.

### DESCRIPTION OF THE DRAWINGS

FIG. 1 illustrates a schematic structural diagram on a medical robot in a plurality of embodiments of the present disclosure;

FIG. 2 illustrates a sectional view on a pitching adjusting mechanism in a plurality of embodiments of the present disclosure;

FIG. 3 illustrates a schematic structural diagram of a cross slide table in a plurality of embodiments of the present disclosure;

FIG. 4 illustrates a schematic diagram of a working state of a surgical auxiliary positioning system in a plurality of embodiments of the present disclosure;

FIG. 5 illustrates a schematic structural diagram of a first adjusting module of FIG. 4;

FIG. 6 illustrates a structural block diagram on the surgical auxillary positioning system of FIG. 4;

FIG. 7 illustrates a schematic diagram of an assembly structure on a surgical auxiliary positioning system and a surgical bed in a plurality of embodiments of the present disclosure;

FIG. 8 illustrates a schematic structural diagram on a first adjusting module of FIG. 7;

FIG. 9 illustrates a flowchart on a control method for a surgical auxillaryauxilary positioning system in a plurality of embodiments of the present disclosure.

### SPECIFIC IMPLEMENTATIONS

For making the objectives, technical solutions and advantages of the present disclosure more clear, the technical solutions of the present disclosure will be clearly and completely described below in conjunction with the accompanying drawings in the present disclosure. Apparently, the described embodiments are some rather than all of the embodiments of the present disclosure. Based on the embodiments of the present invention, other various embodiments obtained by those of ordinary skill in the art without making creative efforts fall within the scope of protection of the present invention. Unless otherwise defined, technical or scientific terms used in the present disclosure are to be given their ordinary meaning as understood by those of ordinary skill in the art to which the present disclosure belongs. As used herein, words such as “comprise” mean that elements or items appearing before the word encompass elements or items listed after the word and equivalents thereof, but do not exclude other elements or items.

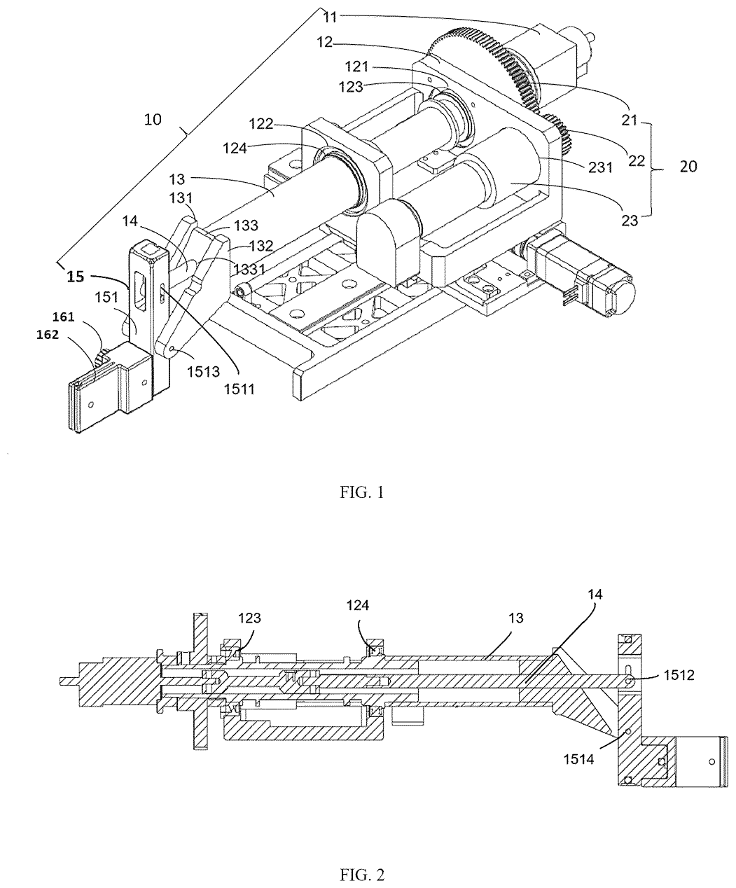

With reference to FIG. 1, a medical robot includes a single-arm mechanism (not shown in the figure), where the single-arm mechanism (not shown in the figure) includes a pitching adjusting mechanism 10 and a deflection adjusting mechanism 20, the pitching adjusting mechanism 10 includes a pitching motor 11, a support frame 12, a main rotating shaft 13, a telescopic pull rod 14 and a pitching frame 15, the pitching frame 15 includes a hinge portion 151, the main rotating shaft 13 is movably connected to the support frame 12, the telescopic pull rod 14 penetrates the main rotating shaft 13, a tail end of the telescopic pull rod 14 is hinged to an upper end of the hinge portion 151, a lower end of the hinge portion 151 is hinged to a tail end of the main rotating shaft 13, an end of the telescopic pull rod 14 away from the hinge portion 151 is connected to the pitching motor 11, and the pitching motor 11 is a telescoping motor and is configured to drive the telescopic pull rod 14 to telescopically slide relative to the main rotating shaft 13 so as to implement pitching movement of the hinge portion 151; and the deflection adjusting mechanism 20 includes a first gear 21, a second gear 22 and a main rotating motor 23, the first gear 21 sleeves an outer wall of one end of the main rotating shaft 13 away from the hinge portion 151, the second gear 22 meshes with the first gear 21, and the main rotating motor 23 is connected to the second gear 22 and configured to drive the second gear 22 to rotate, so as to drive the first gear 21 to rotate, and then to drive the main rotating shaft 13 to rotate to implement deflection movement of the pitching frame 15.

The second gear 22 meshes with the first gear 21, the first gear 21 sleeves an outer wall of the main rotating shaft 13, and the main rotating motor 23 connected to the second gear 22 may drive the first gear 21 by means of the second gear 22, to drive the main rotating shaft 13 to rotate so as to implement deflection movement of the pitching frame 15, such that the deflection adjusting mechanism implements the deflection movement of the pitching frame 15 by means of transmission of the main rotating shaft 13 of the pitching adjusting mechanism, so as to improve an integration level, and effectively reduce an occupied space.

The main rotating motor 23 and the pitching motor 11 are both arranged away from one end of the pitching frame 15, and do not hinder the surgical operation.

In some embodiments, the first gear 21 has a larger outer diameter than the second gear 22.

In some embodiments, the outer diameter of the first gear 21 and the outer diameter of the second gear 22 are different, then a transmission ratio is smaller, and during rotation, the number of turns of the first gear 21 is less than that of the second gear 22, such that when the second gear 22 is rotated, the first gear 21 rotates slower, and precision may be better improved.

With reference to FIG. 1, the support frame 12 includes a first hole 121 and a second hole 122, and the main rotating shaft 13 penetrates the first hole 121 and the second hole 122.

With reference to FIGS. 1 and 2, a first bearing 123 is arranged in the first hole 121, a second bearing 124 is arranged in the second hole 122, and the main rotating shaft 13 penetrates the first bearing 123 and the second bearing 124.

With reference to FIG. 1, the support frame 12 includes a third hole 231 and a fourth hole (no shown in the figure), the main rotating motor 23 penetrates the third hole 231 and the fourth hole (no shown in the figure), and the support frame 12 may fix and support the main rotating motor 23.

With reference to FIGS. 1 and 2, one end of the hinge portion 151 includes a first through hole 1511 and a first pin 1512 that penetrate two opposite walls of the hinge portion 151, a tail end of the telescopic pull rod 14 is connected to the first pin 1512, and the first pin 1512 is located in the first through hole 1511.

With reference to FIG. 1, one end of the main rotating shaft 13 includes a first triangular plate 131, a second triangular plate 132 and a connection plate 133, the first triangular plate 131 and the second triangular plate 132 are arranged oppositely and are connected by means of the connection plate 133, a fifth hole 1331 is provided in the connection plate 133, the telescopic pull rod 14 penetrates the fifth hole 1331, a sixth hole (no shown in the figure) is provided in one end of the first triangular plate 131, a seventh hole (no shown in the figure) is provided in one end of the second triangular plate 132; and the other end of the hinge portion 151 includes a second pin 1513 and is provided with a second through hole 1514, the second pin 1513 is located between the first triangular plate and the second triangular plate to communicate the sixth hole (no shown in the figure), the second through hole 1514 and the seventh hole (no shown in the figure) sequentially, and the second pin 1513 is located in a channel where the sixth hole (no shown in the figure), the second through hole 1514 and the seventh hole (no shown in the figure) are in communication, so as to hinge the hinge portion 151 to one end of the main rotating shaft 13.

With reference to FIG. 1, the medical robot as shown in FIG. 1 further includes the clamping module consisting of an operation portion 162 and a knob 161, the operation portion 162 is provided with a hole, the hinge portion 151 is provided with a hole, and the knob 161 is screwed into the hole provided in the operation portion 162 and the hole provided in the hinge portion 151 to tighten the operation portion 162 and the hinge portion 151.

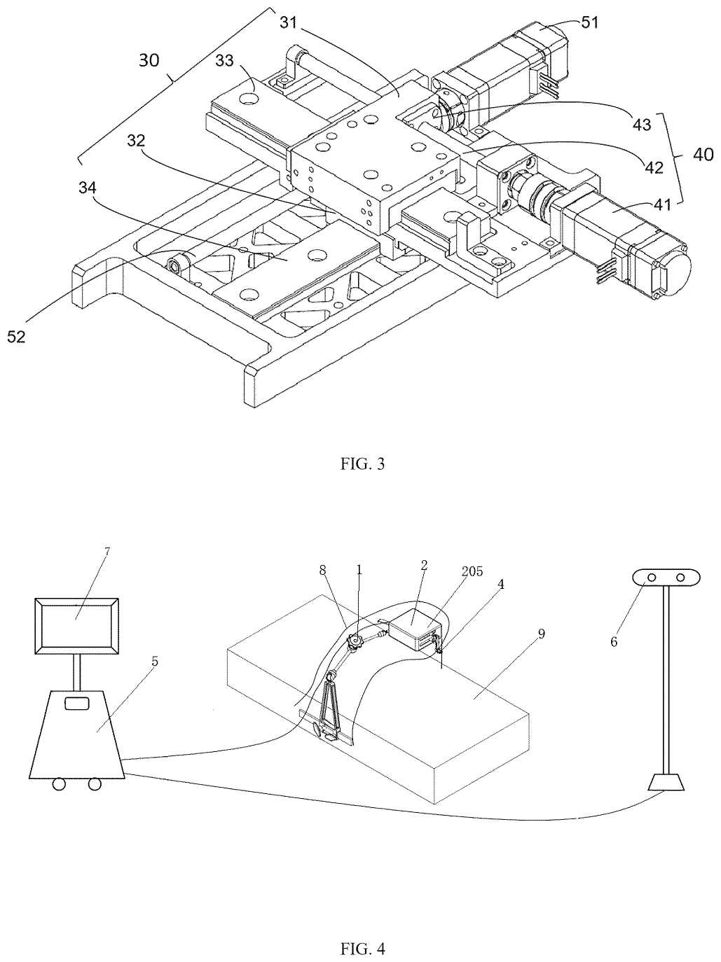

The medical robot further includes a cross slide table. With reference to FIG. 3, the cross slide table includes a rail mechanism 30, the rail mechanism 30 includes a first slider 31, a second slider 32, a first rail 33 and a second rail 34, the first rail 33 and the second rail 34 are located on a horizontal plane, the first rail 33 and the second rail 34 are perpendicular to each other, the first slider 31 is slidably connected to the first rail 33, the first rail 33 is fixedly connected to the second slider 32, and the second slider 32 is slidably connected to the second rail 34 and configured to drive the first rail 33 to slide along the second rail 34.

With reference to FIG. 3, the cross slide table further includes a first screw mechanism 40, the first screw mechanism 40 includes a first electric motor 41, a first screw 42 and a first nut 43, the first electric motor 41 is connected to one end of the first screw 42, the first screw 42 is in threaded connection to the first nut 43, the first slider 31 is fixedly connected to the first nut 43, and the first electric motor 41 is configured to drive the first screw 42 to rotate so as to drive the first slider 31 to move along the first rail 33.

With reference to FIG. 3, the cross slide table further includes a second screw mechanism (not shown in the figure), the second screw mechanism (not shown in the figure) includes a second electric motor 51, a second screw 52 and a second nut (not shown in the figure), the second electric motor 51 is connected to one end of the second screw 52, the second screw 52 is in threaded connection to the second nut (not shown in the figure), the first slider 31 is fixedly connected to the second nut (not shown in the figure), and the second electric motor 51 is configured to drive the second screw 52 to rotate so as to drive the second slider 32 to move along the second rail 34.

The medical robot of the present disclosure includes the cross slide table as shown in FIG. 3 and the single-arm mechanism including the pitching adjusting mechanism 10 and the deflection adjusting mechanism 20 as shown in FIG. 1. The cross slide table includes the rail mechanism 30, the first screw mechanism 40 and the second screw mechanism which includes the second electric motor 51, the second screw 52 and the second nut, and the single-arm mechanism may be driven to move in the front-back and left-right degrees of freedom. The pitching adjusting mechanism 10 and the deflection adjusting mechanism 20 above may drive the pitching frame 15 of the single-arm mechanism to do two-degree-of-freedom motion of pitching and deflection, such that the medical robot of the present application may implement four-degree-of-freedom motion of front-back, left-right, pitching and deflection.

The embodiments of the present disclosure further provide a surgical auxillary positioning system.

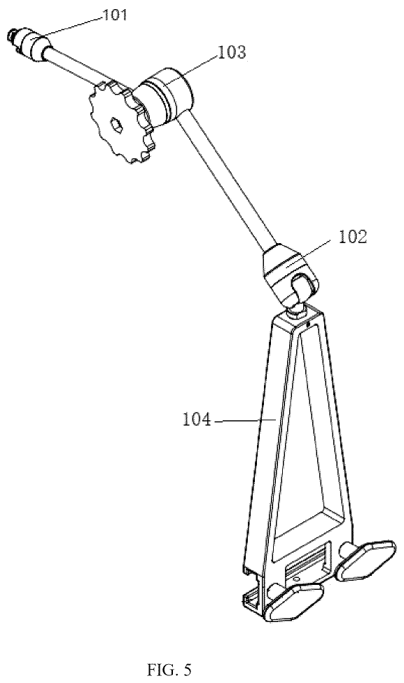

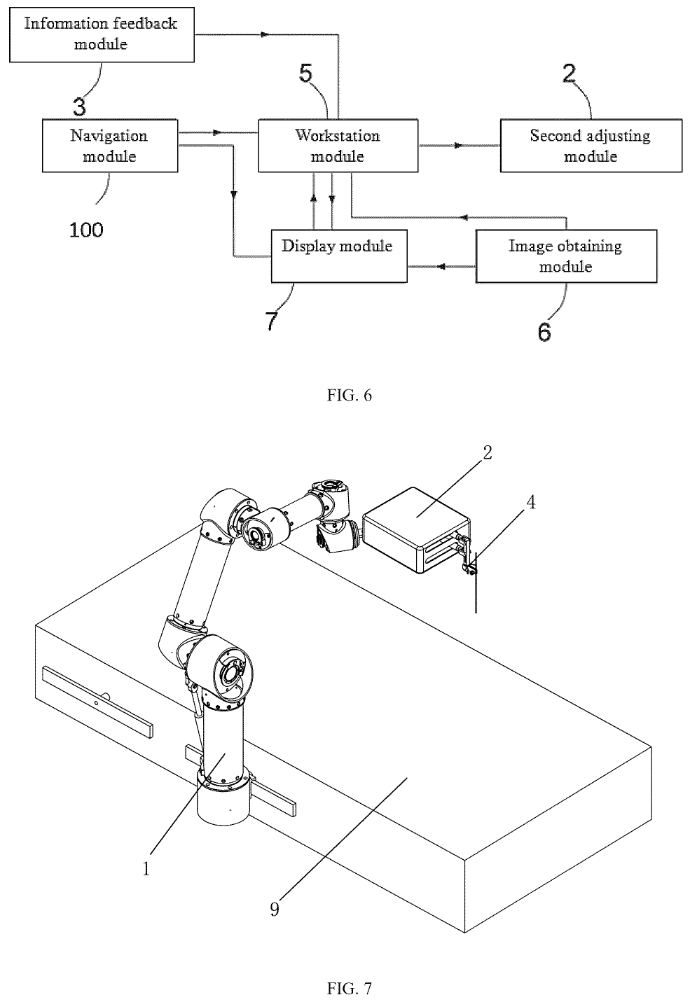

With reference to FIGS. 4-7, the surgical auxillary positioning system as shown in FIG. 4 includes a first adjusting module 1, a second adjusting module 2, an information feedback module 3, a clamping module 4, a workstation module 5, a navigation module 100, an image capturing module 6 and a display module 7, where the second adjusting module 2 is arranged at one end of the first adjusting module 1, the first adjusting module 1 is configured to adjust a working area of the second adjusting module 2, the clamping module 4 is arranged on the second adjusting module 2, the second adjusting module 2 is configured to adjust a position and an angle of the clamping module 4, and the clamping module 4 is configured to fix an surgery execution structure; the information feedback module 3 is electrically connected to the workstation module 5, the information feedback module 3 is arranged on the second adjusting module 2, and the information feedback module 3 is configured to determine working area information of the second adjusting module 2 and transmit the working area information to the workstation module 5; the image capturing module 6 is electrically connected to the display module 7 and the workstation module 5 separately, and the image capturing module 6 obtains image information of a surgical object and transmits the image information to the display module 7 and the workstation module 5; the navigation module 100 is electrically connected to the display module 7 and the workstation module 5 separately, the navigation module 100 is arranged on the clamping module 4 and the surgical object, and the navigation module 100 collects position information of the surgical object and the clamping module 4 and transmits the position information to the display module 7 and the workstation module 5; the display module 7 is electrically connected to the workstation module 5, and the display module 7 receives and displays the image information and the position information; the workstation module 5 is electrically connected to the second adjusting module 2, the workstation module 5 receives the image information and the position information, the workstation module 5 establishes a working path according to the image information, and the workstation module 5 compares the position information with the working path to obtain adjusting information; and the workstation module 5 receives the working area information, and the workstation module 5 outputs an adjusting instruction according to the adjusting information and the working area information to control the second adjusting module 2 to be turned on or off, and determines whether to adjust the first adjusting module 1 according to the adjusting instruction.

In some embodiments, the second adjusting module 2 is the single-arm mechanism including the pitching adjusting mechanism 10 and the deflection adjusting mechanism 20 as shown in FIG. 1.

In some embodiments, the operation portion 162 and the knob 161 as shown in FIG. 1 form the clamping module 4.

In some embodiments, with reference to FIGS. 4 and 7, the surgical auxillary positioning system as shown in FIG. 4 is arranged on a surgical bed 9.

In some embodiments, with reference to FIGS. 4, 5 and 7, the first adjusting module 1 includes a first connection rod 101, a second connection rod 102, a connector 103 and a base 104, where the base 104 is slidably arranged on the surgical bed 9, one end of the first connection rod 101 is rotationally arranged on the base 104, the connector 103 is arranged at the other end of the first connection rod 101, the second connection rod 102 is arranged on the connector 103, the first connection rod 101 and the second connection rod 102 rotate relative to each other by means of the connector 103, the second adjusting module 2 is rotationally arranged at one end of the second connection rod 102 away from the connector 103, the first connection rod 101 may rotate relative to the base 104 freely, the second adjusting module 2 may rotate relative to the second connection rod freely, and the connector 103 may fix an angle between the first connection rod 101 and the second connection rod 102, and the connector 103 may further fix an angle of the first connection rod 101 relative to the base 104 and fix an angle of the second adjusting module relative to the second connection rod 102. During use, the clamping module 4 may be driven to reciprocate relative to the surgical bed 9 by adjusting a position of the base 104 on the surgical bed 9, the base 104 is fixed after adjustment, a fixing state of the first connection rod 101, the second connection rod 102 and the second adjusting module 2 is released by means of the connector 103, the angle of the first connection rod 101 relative to the base 104 is adjusted, the angle of the second connection rod 102 relative to the first connection rod 101 is adjusted, the angle of the second adjusting module 2 relative to the second connection rod 102 is adjusted, and then the first connection rod 101, the second connection rod 102 and the second adjusting module 2 are fixed by means of the connector 103, such that the working area is adjusted, the use is convenient, and the area is wide.

In some embodiments, the clamping module 4 is a clamping frame, the clamping module 4 is configured to fix and guide a surgical instrument, the surgical instrument is one of a puncture needle, an ablation needle or a transforaminal endoscope, the surgical instrument may be manually used for surgery by means of the clamping module 4, alternatively, a driving unit is fixedly arranged on the clamping module 4, the surgical instrument is fixed to a movable end of the driving unit, the driving unit is electrically connected to the workstation module 5, and under automatic control of the workstation module 5, the driving unit is controlled to be turned on or off to automatically perform surgery.

In some embodiments, the image capturing module 6 includes a camera configured to obtain an external contour and a photographing device configured to obtain a perspective image, and the photographing device is one of a computed tomography (CT), magnetic resonance imaging (MRI), an ultrasound imaging device, or an X-ray examination apparatus.

In some embodiments, the navigation module 100 is a positioning device, and the navigation module 100 is configured to determine the position information of the clamping module 4 and the surgical object.

In some embodiments, the workstation module 5 is a host computer.

In some embodiments, the display module 7 is a display, and the display module 7 is arranged on the workstation module 5.

In some embodiments, with reference to FIGS. 4-7, the workstation module 5 establishes a surgical path based on artificial intelligence or operator settings according to the image information, adjusts the first adjusting module 1 and the second adjusting module 2 according to the surgical path and the current position of the clamping module 4, since an adjustable distance of the clamping module 4 under automatic control of the second adjusting module 2 is limited, the workstation module 5 obtains adjusting information according to the surgical path and the position information, the workstation module 5 may determine whether to adjust the first adjusting module 1 based on the artificial intelligence or the operator according to the adjusting information and the working area information, a distance indicated by the adjusting information is the moving direction and distance of the clamping module 4, the area covered by the working area information is a position where the clamping module 4 may reach in the current state, under the condition that the first adjusting module 1 needs adjustment, the base 104 and/or the connector 103 may be adjusted, and under the condition that the first adjusting module 1 needs no adjustment, the second adjusting module 2 is directly controlled by means of the workstation module 5.

In some embodiments, the first adjusting module 1 may be adjusted preliminarily by means of a surgical site of the surgical object before surgery, so as to avoid adjustment of the first adjusting module 1 or shorten an adjusting amplitude of the first adjusting module 1 during determination, and time is shortened.

Furthermore, under the condition that the first adjusting module 1 needs adjustment, the workstation module 5 sends out an adjusting signal and transmits the adjusting information to the display module 7 for display, and the operator adjusts the first adjusting module 1 according to the observed information.

In some embodiments of the present disclosure, the surgical auxiliary positioning system further includes an image processing module, where the image processing module is electrically connected to the image capturing module 6, the display module 7 and the workstation module 5 separately, the image processing module is configured to fuse different image information obtained by the image capturing module 6, and the image processing module transmits the fused image information to the image capturing module 6, the display module 7 and the workstation module 5.

Specifically, in this embodiment, since the image information of the surgical object changes at different moments or different states, the image processing module fuses the different image information by capturing the image information at different moments or different states, so as to perform the surgery more accurately.

In some embodiments of the present disclosure, the surgical auxillary positioning system further includes an image analysis module, where the image analysis module is connected to the image capturing module 6, the image processing module, the display module 7 and the workstation module 5 separately, the image processing module or the image capturing module 6 transmits the image information to the image analysis module, the image analysis module receives the image information, the image analysis module analyzes positions of a tissue structure and a focus area in the image information and delineates boundaries of the tissue structure and the focus area, and the image analysis module transmits the delineated image information to the workstation module 5 and the display module 7.

In some embodiments, the image analysis module is arranged on the workstation module 5, the image analysis module is electrically connected to the image capturing module 6, the image processing module, the display module 7 and the workstation module 5 separately, image information of a tissue structure and image information of a normal human body are stored in the image analysis module, and the image analysis module distinguishes the tissue structure part and the focus area part of the surgical object by comparing the obtained image information with the stored image information, delineates the boundaries of the tissue structure part and the focus area part, and transmits the delineated image information to the workstation module 5, such that the operator may conveniently observe and make a solution.

In some embodiments of the present disclosure, the surgical auxillary positioning system further includes a comparison module, a storage module and a calling module, where the comparison module is electrically connected to the workstation module 5, the workstation module 5 transmits the delineated image information and the working path to the comparison module, the comparison module receives the delineated image information and the working path, the comparison module is configured to compare the working path with the position of the tissue structure and the position of the focus area separately to obtain a comparison result, and the comparison module transmits the comparison result to the workstation module 5, the storage module is electrically connected to the workstation module 5, the workstation module 5 receives the comparison result, the workstation module 5 transmits the working path to the storage module or deletes the working path according to the comparison result, and the storage module receives and stores the working path; and the calling module is electrically connected to the storage module and the workstation module 5 separately, and the calling module calls the working path stored by the storage module and transmits the working path to the workstation module 5.

In some embodiments, the comparison module, the storage module and the calling module are all arranged on the workstation module 5, the workstation module 5 transmits the delineated image information and the working path to the comparison module, the comparison module is configured to compare whether the working path is crossed with the tissue structure and the focus area in the image information and feed back a comparison result to the workstation module 5, the workstation module 5 analyzes feasibility of the working path according to the comparison result, transmits the feasible solution to the storage module for storage, and repeats comparison and storage of a plurality of paths, to obtain a plurality of feasible solutions, one stored path is called for surgery by means of the calling module during the surgery, and in this way, the surgical time is saved by making a surgical solution before the surgery and selecting according to needs during the surgery.

In some embodiments of the present disclosure, the single-arm mechanism includes an execution module, the execution module is arranged on the clamping module 4, the execution module is connected to the workstation module 5, and the workstation module 5 is configured to control the execution module to be turned on or off. In some embodiments, the execution module is a linear motor, the execution module is arranged on the clamping module 4, a movable end of the execution module is connected to the clamping module 4, the clamping module 4 clamps an execution instrument, and the execution module may drive the execution instrument to move in a direction driven by the execution module, such that the direction and depth are convenient to control, and errors caused by external reasons are eliminated.

In some embodiments of the present disclosure, the navigation module 100 includes a reference target and an optical target; and the reference target is arranged on the surgical object, and the optical target is arranged on the clamping module 4.

In some embodiments, before surgery, the reference target is fixed on the surgical object or the surgical bed 9, when the reference target is fixed on the surgical bed 9, relative position information of the surgical object and the surgical bed 9 may be determined according to original image information, that is, the reference target may be regarded as an origin of coordinates and three-dimensional position information of the surgical object may be determined. The optical target is arranged on the clamping module 4, the position information of the clamping module 4 relative to the reference target may be determined directly by means of the optical target, and the position information of the execution module relative to the surgical object may be determined indirectly. In this way, the position information of the clamping module 4 relative to the surgical object may be conveniently obtained, and the position of the clamping module 4 may be conveniently adjusted.

Furthermore, in some embodiments of the present disclosure, the navigation module 100 includes an optical target and an optical camera, where the optical target is arranged on the clamping module 4, the optical camera is arranged corresponding to the clamping module 4, and the optical camera recognizes the optical target and determines relative position information of the optical target and the surgical object.

In some embodiments of the present disclosure, with reference to FIG. 4, the surgical auxillary positioning system further includes an isolation layer structure 8, where the isolation layer structure 8 is arranged outside the first adjusting module 1 and the second adjusting module 2.

In some embodiments, the isolation layer structure 8 is a sterile bag, the isolation layer structure 8 sleeves the first adjusting module 1 and the second adjusting module 2, and since transmission structures are arranged in the first adjusting module 1 and the second adjusting module 2, contamination may be reduced by means of the isolation layer structure 8, so as to improve safety of surgery.

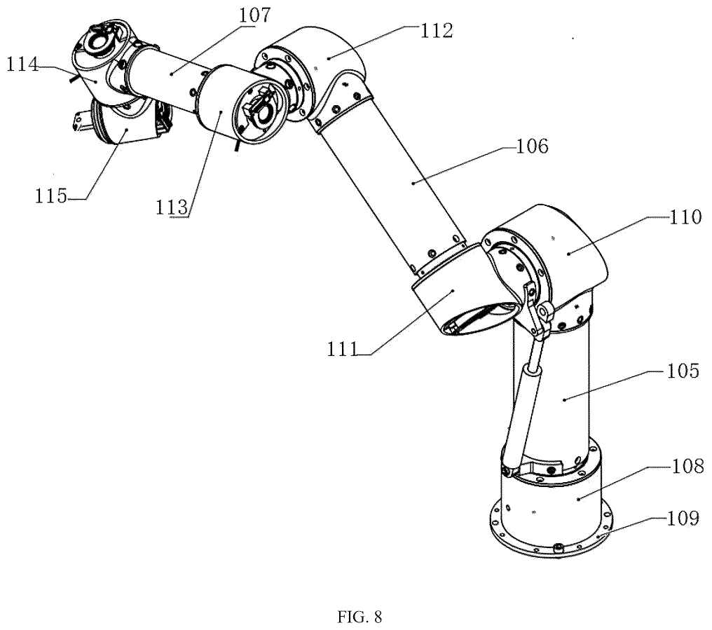

In some embodiments of the present disclosure, with reference to FIGS. 4, 5, 7 and 8, the first adjusting module 1 includes a column 105, a large arm 106 and a small arm 107, where the column 105 is movably connected to the base 109 by means of a first rotary joint 108, the column 105 is movably connected to the large arm 106 by means of a second rotary joint 110 and a third rotary joint 111, the large arm 106 is movably connected to the small arm 107 by means of a fourth rotary joint 112 and a fifth rotary joint 113, and a tail end of the small arm 107 is movably connected to a sixth rotary joint 114 and a seventh rotary joint 115 in sequence, such that the first adjusting module 1 has seven degrees of freedom, multi-degree-of-freedom adjustment and rapid positioning of any posture may be implemented, space positioning is flexible, and positioning is convenient and rapid. During use, the base 109 is slidably arranged on the surgical bed 9, and the second adjusting module 2 is arranged on the seventh rotary joint 115.

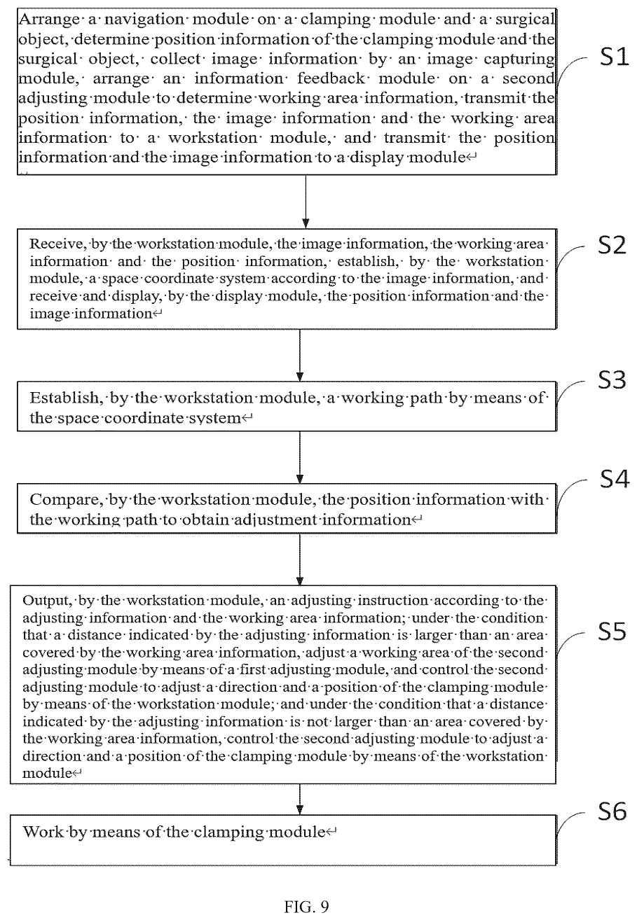

In some embodiments of the present disclosure, with reference to FIGS. 4, 6 and 9, a control method for a surgical auxillary positioning system includes:

S1, arrange the navigation module 100 on the clamping module 4 and the surgical object, determine the position information of the clamping module 4 and the surgical object, collect the image information by means of the image capturing module 6, arrange the information feedback module 3 on the second adjusting module 2 to determine the working area information, transmit the position information, the image information and the working area information to the workstation module 5, and transmit the position information and the image information to the display module 7;

S2, receive, by the workstation module 5, the image information, the working area information and the position information, establish, by the workstation module 5, a space coordinate system by means of the image information, and receive and display, by the display module 7, the position information and the image information;

S3, establish, by the workstation module 5, a working path by means of the space coordinate system;

S4, compare, by the workstation module 5, the position information with the working path to obtain adjustment information;

S5, output, by the workstation module 5, an adjusting instruction according to the adjusting information and the working area information; under the condition that a distance indicated by the adjusting information is larger than an area covered by the working area information, adjust a working area of the second adjusting module 2 by means of the first adjusting module 1, and control the second adjusting module 2 to adjust a direction and a position of the clamping module 4 by means of the workstation module 5; and under the condition that a distance indicated by the adjusting information is not larger than an area covered by the working area information, control the second adjusting module 2 to adjust a direction and a position of the clamping module 4 by means of the workstation module 5; and

S6, work by means of the clamping module 4.

Specifically, in this embodiment, the reference target is arranged on the surgical object or the surgical bed 9, the optical target is arranged on the execution instrument, and the image information is collected by the image capturing module 6, such that the relative position information of the surgical object and the execution instrument may be determined, and it is guaranteed that the execution instrument performs execution according to a preset solution when surgery is executed. The information feedback module 3 determines the working area information, and a position area where the clamping module 4 may reach in the current state may be known, then the workstation module 5 establishes a space coordinate system by taking the reference target or any point as a reference original point according to the image information, the working area information and the position information, such that space three-dimensional coordinate information of the surgical object and the clamping module 4 may be known. The image information and the position information are displayed by the display module 7, and the three-dimensional coordinate information may be transmitted to the workstation module 5 to be displayed during use, such that the operator may select a target position and observe conveniently, then the workstation module 5 establishes a working path based on the artificial intelligence or operation of the operator according to the information, after the working path is obtained, the position of the clamping module 4 in the current state needs to be adjusted, adjustment information is computed by the workstation module 5, and whether the first adjusting module 1 needs adjustment is determined according to the adjusting information and the working area information. Under the condition that the first adjusting module 1 needs adjustment, the working area information and the position information need to be confirmed again after adjustment and are compared with the working path, and the workstation module 5 controls the second adjusting module 2 to adjust the position of the clamping module 4. Under the condition that the first adjusting module 1 needs no adjustment, the workstation module 5 directly controls the second adjusting module 2 to adjust the position of the clamping module 4, and then the surgery is performed by means of the clamping module 4.

In some embodiments of the present disclosure, between S5 and S6, the following steps are further included: determine, by the navigation module 100, again the position information of the clamping module 4 adjusted in S5 and transmit the position information to the workstation module 5, receive, by the workstation module 5, the adjusted position information, compare, by the workstation module 5, the adjusted position information with a working path, execute S4 under the condition that the position information does not coincide with the working path, and execute S6 under the condition that the position information coincides with the working path.

Specifically, in this embodiment, by means of the arrangement, the position of the execution module adjusted in S5 is compared with the position indicated by the working path, it is guaranteed that the execution module is adjusted in place, and the adjustment accuracy is guaranteed.

In some embodiments of the present disclosure, between S5 and S6, the control method further includes the following steps:

S51, collect image information again by means of the image capturing module 6, and transmit the image information to the workstation module 5;

S52, receive, by the workstation module 5, the image information collected again, compare, by the workstation module 5, the image information collected again with the image information fused in S2 or the image information collected in S1, and computed, by the workstation module 5, a deviation between the image information collected again and the image information fused in S2 or the image information collected in S1; and

S53, under the condition that the deviation exists, regard the deviation as adjusting information and execute S5, and under the condition that no deviation exists, execute S6.

Specifically, in this embodiment, the image information of the surgical object is confirmed again before S6, so as to add the movement information to the surgical solution after the surgical object moves, and further to make corresponding adjustment for the surgical path.

Although implementation modes of the present disclosure have been described in detail above, it will be apparent to those skilled in the art that various modifications and variations can be made to these implementation modes. However, it is to be understood that such modifications and variations fall within the scope and spirit of the present disclosure as set forth in the claims. Moreover, the present disclosure described herein can have other implementation modes and be implemented or carried out in various ways.

What is claimed is:

1. A medical robot, comprising a single-arm mechanism, wherein the single-arm mechanism comprises a pitching adjusting mechanism and a deflection adjusting mechanism;
the pitching adjusting mechanism comprises a pitching motor, a support frame, a main rotating shaft, a telescopic pull rod and a pitching frame, the pitching frame comprises a hinge portion, the main rotating shaft is movably connected to the support frame, the telescopic pull rod is located in the main rotating shaft, a first end of the telescopic pull rod is hinged to a first end of the hinge portion, a second end of the hinge portion is hinged to an end of the main rotating shaft, a second end of the telescopic pull rod is connected to the pitching motor, and the pitching motor is configured to drive the telescopic pull rod to telescopically slide relative to the main rotating shaft to implement pitching movement of the hinge portion; and
the deflection adjusting mechanism comprises a first gear, a second gear and a main rotating motor, the first gear sleeves an outer side of the main rotating shaft, the second gear meshes with the first gear, and the main rotating motor is connected to the second gear and configured to drive the second gear to rotate to drive the first gear to rotate and further to drive the main rotating shaft to rotate to implement deflection movement of the pitching frame.

2. The medical robot according to claim 1, wherein the first gear has a larger outer diameter than the second gear.

3. The medical robot according to claim 1, wherein the support frame comprises a first hole and a second hole, and the main rotating shaft penetrates the first hole and the second hole.

4. The medical robot according to claim 3, wherein a first bearing is arranged in the first hole, a second bearing is arranged in the second hole, and the main rotating shaft penetrates the first bearing and the second bearing.

5. The medical robot according to claim 1, wherein the support frame comprises a third hole and a fourth hole, and the main rotating motor penetrates the third hole and the fourth hole.

6. The medical robot according to claim 1, wherein the pitching motor is a telescoping motor.

7. The medical robot according to claim 1, wherein the first end of the hinge portion comprises a first through hole and a first pin, the first through hole and the first pin penetrate two opposite walls of the hinge portion, the first end of the telescopic pull rod is connected to the first pin, and the first pin is located in the first through hole.

8. The medical robot according to claim 7, wherein the end of the main rotating shaft comprises a first triangular plate, a second triangular plate and a connection plate, the first triangular plate and the second triangular plate are arranged oppositely and are connected by means of the connection plate, a fifth hole is provided in the connection plate, the telescopic pull rod penetrates the fifth hole, a sixth hole is provided in an end of the first triangular plate, and a seventh hole is provided in an end of the second triangular plate; and
the second end of the hinge portion comprises a second pin and is provided with a second through hole, the second pin is located between the first triangular plate and the second triangular plate to communicate the sixth hole, the second through hole and the seventh hole sequentially, and the second pin is located in a channel where the sixth hole, the second through hole and the seventh hole are in communication to hinge the hinge portion to the end of the main rotating shaft.

9. The medical robot according to claim 1, further comprising a cross slide table, wherein the cross slide table comprises a rail mechanism, the rail mechanism comprises a first slider, a second slider, a first rail and a second rail, the first rail and the second rail are located on a horizontal plane, the first rail and the second rail are perpendicular to each other, the first slider is slidably connected to the first rail, the first rail is fixedly connected to the second slider, and the second slider is slidably connected to the second rail and configured to drive the first rail to slide along the second rail.

10. The medical robot according to claim 9, wherein the cross slide table further comprises a first screw mechanism, the first screw mechanism comprises a first electric motor, a first screw and a first nut, the first electric motor is connected to an end of the first screw, the first screw is in threaded connection to the first nut, the first slider is fixedly connected to the first nut, and the first electric motor is configured to drive the first screw to rotate to drive the first slider to move along the first rail.

11. The medical robot according to claim 10, wherein the cross slide table further comprises a second screw mechanism, the second screw mechanism comprises a second electric motor, a second screw and a second nut, the second electric motor is connected to an end of the second screw, the second screw is in threaded connection to the second nut, the first slider is fixedly connected to the second nut, and the second electric motor is configured to drive the second screw to rotate to drive the second slider to move along the second rail.

12. A surgical auxiliary positioning system, comprising a first adjusting module, a second adjusting module, an information feedback module, a clamping module, a workstation module, a navigation module, an image capturing module and a display module, wherein the second adjusting module is the single-arm mechanism of the medical robot according to claim 1;
the second adjusting module is arranged at an ene-end of the first adjusting module, the first adjusting module is configured to adjust a working area of the second adjusting module, the clamping module is arranged on the second adjusting module, the second adjusting module is configured to adjust a position and an angle of the clamping module, and the clamping module is configured to fix an surgery execution structure;
the information feedback module is electrically connected to the workstation module, the information feedback module is arranged on the second adjusting module, and the information feedback module is configured to determine working area information of the second adjusting module and transmit the working area information to the workstation module;
the image capturing module is electrically connected to the display module and the workstation module separately, and the image capturing module obtains image information of a surgical object and transmits the image information to the display module and the workstation module;
the navigation module is electrically connected to the display module and the workstation module separately, the navigation module is arranged on the clamping module and the surgical object, and the navigation module collects position information of the surgical object and the clamping module and transmits the position information to the display module and the workstation module;
the display module is electrically connected to the workstation module, and the display module receives and displays the image information and the position information;
the workstation module is electrically connected to the second adjusting module, the workstation module receives the image information and the position information, the workstation module establishes a working path according to the image information, and the workstation module compares the position information with the working path to obtain adjusting information; and
the workstation module receives the working area information, and the workstation module outputs an adjusting instruction according to the adjusting information and the working area information to control the second adjusting module to be turned on or off, and determines whether to adjust the first adjusting module according to the adjusting instruction.

13. The surgical auxillary positioning system according to claim 12, further comprising an image processing module, wherein
the image processing module is electrically connected to the image capturing module, the display module and the workstation module separately, the image processing module is configured to fuse different image information obtained by the image capturing module, the image processing module transmits fused image information to the image capturing module, the display module and the workstation module.

14. The surgical auxillary positioning system according to claim 13, further comprising an image analysis module, wherein
the image analysis module is connected to the image capturing module, the image processing module, the display module and the workstation module separately, the image processing module or the image capturing module transmits the image information to the image analysis module, the image analysis module receives the image information, the image analysis module analyzes positions of a tissue structure and a focus area in the image information and delineates boundaries of the tissue structure and the focus area, and the image analysis module transmits delineated image information to the workstation module and the display module.

15. The surgical auxillary positioning system according to claim 14, further comprising a comparison module, a storage module and a calling module, wherein
the comparison module is electrically connected to the workstation module, the workstation module transmits the delineated image information and the working path to the comparison module, the comparison module receives the delineated image information and the working path, the comparison module is configured to compare the working path with the position of the tissue structure and the position of the focus area separately to obtain a comparison result, and the comparison module transmits the comparison result to the workstation module;
the storage module is electrically connected to the workstation module, the workstation module receives the comparison result, the workstation module transmits the working path to the storage module or deletes the working path according to the comparison result, and the storage module receives and stores the working path; and
the calling module is electrically connected to the storage module and the workstation module separately, and the calling module calls the working path stored by the storage module and transmits the working path to the workstation module.

16. The surgical auxillary positioning system according to claim 15, wherein the single-arm mechanism comprises an execution module, the execution module is arranged on the clamping module, the execution module is connected to the workstation module, and the workstation module is configured to control the execution module to be turned on or off.

17. The surgical auxillary positioning system according to claim 12, wherein the navigation module comprises a reference target and an optical target; and
the reference target is arranged on the surgical object, and the optical target is arranged on the clamping module.

18. The surgical auxillary positioning system according to claim 12, further comprising an isolation layer structure arranged outside the first adjusting module and the second adjusting module.

19. A control method for the surgical auxillary positioning system according to claim 12, comprising:
S1, arranging the navigation module on the clamping module and the surgical object, determining the position information of the clamping module and the surgical object, collecting the image information by the image capturing module, arranging the information feedback module on the second adjusting module to determine the working area information, transmitting the position information, the image information and the working area information to the workstation module, and transmitting the position information and the image information to the display module;
S2, receiving, by the workstation module, the image information, the working area information and the position information, establishing, by the workstation module, a space coordinate system according to the image information, and receiving and displaying, by the display module, the position information and the image information;
S3, establishing, by the workstation module, the working path by means of the space coordinate system;
S4, comparing, by the workstation module, the position information with the working path to obtain adjustment information;
S5, outputting, by the workstation module, the adjusting instruction according to the adjusting information and the working area information; under a condition that a distance indicated by the adjusting information is larger than an area covered by the working area information, adjusting the working area of the second adjusting module by means of the first adjusting module, and controlling the second adjusting module to adjust a direction and the position of the clamping module by means of the workstation module; and under a condition that the distance indicated by the adjusting information is less than or equal to the area covered by the working area information, controlling the second adjusting module to adjust the direction and the position of the clamping module by means of the workstation module; and
S6, working by means of the clamping module.
